SEQUENCE LISTING (3 sequences)

SEQ ID NO: 1 - protein, 13 aa, Dermatophoides pteronyssinus
IIHRGKPFQLEAV

SEQ ID NO: 2 - protein, 13 aa, Dermatophoides pteronyssinus
IIHRGKAFALEAV

SEQ ID NO: 3 - protein, 114 aa, Flammalina velutipes
SATSLTFQLAVLVKKIDFDYTPNWGRGTPSSYIDNLTFPKVLTDKKYSYRVVVNGSDLGVESNFAVTPSGGQTINFLQYNKGYGVADTKTIQVFVVIPDTGNSEEYIIAEWKKT

(19) United States
(12) Patent Application Publication
(10) Pub. No.: US 2004/0071718 A1
(43) Pub. Date: Apr. 15, 2004

(54) Local nasal immunotherapy for allergen-induced airway inflammation

(76) Inventor: Jaw-Ji Tsai, Taipei (TW)

Correspondence Address: BIRCH STEWART KOLASCH & BIRCH, PO BOX 747, FALLS CHURCH, VA, 22040-0747, US

(21) Appl. No.: 10/245,514

(22) Filed: Sep. 18, 2002

(51) Int. Cl. A61K 39/00

(52) U.S. Cl. 424/185.1

(57) ABSTRACT

Methods and pharmaceutical compositions for treating allergen-induced airway inflammation. The method includes administering a therapeutically effective amount of a pharmaceutical composition comprising a Dermatophoides pteronyssinus group 2 (Dp2) epitope peptide and a pharmaceutically acceptable carrier to an individual having allergen-induced airway inflammation, in a manner consistent with local nasal immunotherapy.

### BACKGROUND OF THE INVENTION

1. Field of the Invention

The present invention relates to pharmaceutical compositions and methods for the treatment of allergen-induced airway inflammation. More particularly, the present invention relates to pharmaceutical compositions and methods for local nasal immunotherapy of allergen-induced airway inflammation.

2. Description of the Related Arts

Epidemiological studies suggest that between 10 and 20% of the world population exhibits some form of IgE mediated hypersensitivity, which is manifested as asthma, atopic dermatitis, or allergic rhinitis (Platts-Mills TA, et al. J Allergy Clin Immunol 1997; 100: S2-24). A number of studies have shown that sensitivity to house dust mite allergens is the most important risk factor for asthma (Platts-Mills TA, et al. J Allergy Clin Immunol 1997; 100: S2-24; Sporik R, et al. Clin Exp Allergy 1992; 22: 897-906). More than 10 mite allergens have been defined, and the 14-kDa Group 2 allergens (Dp2 and Df2) are considered major allergens because of the fact that 80-90% of patients have specific IgE antibodies to these allergens (Heymann, P W, et al. J Allergy Clin Immunol 1989; 83: 1055-67). Therapy for allergic disease includes allergen avoidance, pharmacotherapy, and allergen-specific immunotherapy (Smith A M, and Chapman M D. In immunotherapy in Asthma, Bousquet, J. and Yssel, H., eds, Marcel Dekker, Inc., New York.1999).

For the control of intractable atopic disease against unavoidable allergens, hyposensitizing therapy, or allergen immunotherapy, has been the major realistic means of control (Noon L. Lancet 1911; 1:1572; Rolland J, and R.O'Hehir. Curr. Opin. Immunol 1998; 10:640-5; Barnes P J. Nature1999; 402 :S31-8). However, the therapy has suffered from a number of problems (Grant J A. New Engl J Med 1979; 300:565-565; Reid M J, et al. J Allergy Clin Immunol 1993; 92:6-15; Korematsu S, et al. J Immunol. 2000; 165:2895-902). Although complications deriving from the impurity of allergens have been overcome by the production of purified recombinant allergens, provocation of possibly life-threatening anaphylactic reactions remains a serious adverse effect. There is still a need for therapies of allergen-induced airway inflammation with improved efficacy, patient compliance and associated risk reduction.

### SUMMARY OF THE INVENTION

It is therefore a primary object of the present invention to provide a method and pharmaceutical composition for treating allergen-induced airway inflammation in an individual. The method of the present invention comprises the steps of: administering an effective amount of a pharmaceutical composition comprising Dermatophoides pteronyssinus group 2 (Dp2) epitope peptide and a pharmaceutically acceptable carrier to an individual having allergen-induced airway inflammation, in a manner consistent with local nasal immunotherapy (LNIT). In one embodiment, the allergen-induced airway inflammation comprises, but is not limited to, allergic asthma, allergic rhinitis, or nasosinusitis. The Dermatophoides pteronyssinus group 2 (Dp2) epitope peptide is Dp2 28-40 or Dp2 28-40A. Dp2 28-40 is encoded from amino acid sequence ID No. 1, and Dp2 28-40A is encoded from amino acid sequence ID No. 2. The pharmaceutical composition further comprises a fungal immunomodulatory protein (FIP) isolated from Flammalina velutipes and the FIP is encoded from amino acid sequence ID No. 3.

In one embodiment, the Dp 2 epitope peptide is in a dosage of 0.1˜10 μg, preferably 1 μg. The FIP is in a dosage of 1˜100 μg, preferably 50 μg.

In another aspect, the pharmaceutical composition of the present invention comprises a Dermatophoides pteronyssinus group 2 (Dp2) epitope peptide; and a pharmaceutically acceptable carrier. In one embodiment, the pharmaceutical composition is in the form of nasal drops, nasal spray, cream or nasal strip. The nasal drops or nasal spray include PBS or normal saline with 10% glycerol as the pharmaceutically acceptable carrier. The materials of the nasal strip include nitrocellulose (NC), polyvinylidene fluoride (PVDF), nylon, filter papers, fabric, cloth, polyethylene, polypropylene, composite fibers, flexible medical grade materials, or the combination of above mentioned materials. The cream includes vaseline as the pharmaceutically acceptable carrier.

The Dermatophoides pteronyssinus group 2 (Dp2) epitope peptide is Dp2 28-40 or Dp2 28-40A. Dp2 28-40 is encoded from amino acid sequence ID No. 1, and Dp2 28-40A is encoded from amino acid sequence ID No. 2. The pharmaceutical composition further comprises a fungal immunomodulatory protein (FIP) isolated from Flammalina velutipes and the FIP is encoded from amino acid sequence ID No. 3.

In one embodiment, the Dp 2 epitope peptide is in a concentration of 0.1˜10 μg/20 μl, preferably 1 μg/20 μ. The FIP is in a concentration of 1˜100 μg/20 μl, preferably 50 μg/20 μl.

### BRIEF DESCRIPTION OF THE DRAWINGS

The present invention will be more fully understood and further advantages will become apparent when reference is made to the following description of the invention and the accompanying drawings in which:

FIG. 1 is a diagram showing a protocol for intraperitoneal (IP) sensitization with recombinant Dp2 (rDp2) and local nasal immunotherapy (LNIT) with Dp2 derived peptides, FIP alone or FIP mixed with Dp2-derived peptides after immunization and subsequent intratracheal (IT) challenge with rDp2. Normal saline(NS), Dexamethasone(DEX), Blomia tropicalis(Bt) were used as control.

FIGS. 2A-2C are diagrams showing the effects of Dp2 28-40 and Dp2 28-40A on Dp2 specific IgG1 (FIG. 2A), IgG2a (FIG. 2B) and IgE (FIG. 2C) production in the sera. *p<0.05 compared with NS.

FIGS. 3A-3D are diagrams showing the effects of Dp2 28-40 and Dp2 28-40A on IL-5 levels in sera (FIG. 3A) and BALF (FIG. 3C) and IFN-Υ levels in sera (FIG. 3B) and BALF (FIG. 3D). *p<0.05 compared with NS.

FIG. 4 is a diagram showing the effects of Dp2 28-40 on free radical production. Free radicals in the BALF were measured immediately after each experiment. *p<0.05 compared with NS.

FIGS. 5A-5B are diagrams showing the effects of Dp2 28-40 and Dp2 28-40A on pulmonary function. The airway hypersensitivity to methacholine measured at both early (FIG. 5A) and late (FIG. 5B) phase was accumulated after each dose of methacholine challenge after rDp2 IT challenge. The percentage changes in Penh were compared between the LNIT mice. *p<0.05 compared with NS.

FIGS. 6A-6F are photographs showing the pathology of different groups of mice after LNIT. The lung tissues were obtained two days after IT with rDp2. FIG. 6A: naive mice, FIG. 6B: NS treated mice; FIG. 6C: DEX treated mice; FIG. 6D: Dp2 28-40 treated mice; FIG. 6E: Dp2 28-40A treated mice; and FIG. 6F: Bt treated mice. (HE stain, ×400).

FIGS. 7A-7C are diagrams showing the effects of FIP and FIP combined with a Dp2 peptide mixture, as regards specific IgG1 (FIG. 7A), IgG2a (FIG. 7B) and IgE (FIG. 7C) production in the sera. *p<0.05 compared with NS.

FIGS. 8A-8B are diagrams showing the effects of FIP alone, and FIP combined with a Dp2 peptide upon pulmonary function amongst test mice. The airway hypersensitivity to methacholine was measured at both relatively early (30 min) (FIG. 8A) and late(24 hr) (FIG. 8B) phases. The proportional (percentage) change in Penh was compared amongst these LNIT mice. *p<0.05 compared with NS.

FIGS. 9A-1˜9F-2 are photographs showing the pathology of different groups of mice following LNIT. The lung tissues were obtained two days subsequent to IT inoculation with rDp2. FIGS. 9A-1, -2: naive mice; FIGS. 9B-1, -2: NS-treated mice; FIGS. 9C-1, -2: DEX-treated mice; FIGS. 9D-1, -2: FIP-fve treated mice; FIGS. 9E-1, -2: Dp2 28-40A+FIP-fve treated mice; and FIGS. 9F-1, -2: Dp2 28-40+FIP-fve treated mice. FIGS. 9A-1, 9B-1, 9C-1, 9D-1, 9E-1 and 9F-1: HE stain, 100×; 9A-2, 9B-2, 9C-2, 9D-2, 9E-2, 9F-2: HE stain, 400×

### DETAILED DESCRIPTION OF THE INVENTION

Allergen-specific immunotherapy is an established form of treatment for atopic allergic diseases (Durham S R, et al. N Engl J Med 1999;341:468-75). Several studies have suggested that successful treatment of this type is associated with alterations to T-cell function by mechanisms such as a change in the T-cell phenotype from Th2 to Th1(Varneey V A, et al. J Clin Invest 1993;92:644-51; Secrist H, et al. J Exp Med 1993;178:123-30; Jutel M, et al. J Immunol 1995;154:4187-94), although unpredictable anaphylactic reactions due to IgE crosslinking limit the usefulness of hyposensitization as well as the dose difficulties associated with the standardizing of protein levels in complex allergic extracts. It has been previously shown that peripheral T-cell tolerance could be induced in naive and primed mice by the subcutaneous injection of peptides from the major cat allergen, Fel d1 (Briner T J, et al. Proc Natl Acad Sci USA 1993;90:7608-12). Short Fel d1-derived peptides have been shown to be directly able to initiate a major histocompatibility complex-restricted, T cell-dependent, late asthmatic reaction (LAR), without the requirement for an early IgE/mast cell-dependent response, amongst cat-allergic asthmatics(Haselden B M, et al. J Exp Med 1999;189:1885-94) . This raises the possibility that short, allergen-derived, peptides could be used as a therapeutic vaccine since they may be able to inhibit T-cell function without cross-linking IgE, thus avoiding the problem of anaphylaxis.

A number of studies have shown that sensitivity to house-dust mite allergens is the most important risk factor for asthmatics (Platts-Mills, T A, et al. J Allergy Clin Immunol 1997;100: S2-24; Sporik R, et al. Clin Exp Allergy 1992;22:897-906). More than ten mite allergens have been previously defined, and the 14 kDa group 2 allergens (Dp2 and Df2) are considered major allergens because 80-90% of patients express a specific IgE antibody to these allergens(Heymann, P W, et al. J Allergy Clin Immunol 1989;83:1055-67). Although the function of Dp2 remains largely unknown, Dp2 is, structurally, a member of the immunoglobulin superfamily (Mueller G A, et al. Biochemistry 1998;37:12707-14). It has been reported that the human peripheral T cell response to Dp2 is primarily caused by two immunodominant regions, these being residues 28-40 and 101-109. The immunological activity of an altered TCR ligand from the immunodominant T cell epitope of Dp2, in which residues at positions 34 and 36 were substituted by alanine, Dp2 28-40A, was demonstrated to induce elevate IFN-γ synthesis at equimolar concentrations as compared with native peptide (Dp2 28-40) (Verhoef A, and Lamb J R. J Immunol 2000; 164:6034-6040). Recently, it has been demonstrated that intradermal administration of short allergen-derived T cell peptide epitopes produce a reduction in the size of cutaneous late phase reactions following whole allergen challenge (Oldfield W L G, et al. J Immunol 2001; 167:1734-39). Local nasal immunotherapy (LNIT) has long been recognized as being capable of controlling the symptoms of seasonal allergic rhinitis in a dose-dependent manner (Malling H J, et al. Local immunotherapy. Allergy. 1998;53:933-44). In one embodiment, the present invention provides a local nasal immunotherapy of Dp2 28-40 or Dp2 28-40A for allergen-induced airway inflammation and improves the effectiveness of hyposensitization without adverse effects.

A fungal immunomodulatory protein (FIP) isolated from the edible golden needle mushroom (Flammalina velutipes) has been reported to possess immunomodulatory activity. According to a study of Ko et al., the FIP can not only suppress a systemic anaphylactic reaction and the local swelling of mouse footpads, but it may also enhance the transcriptional expression of interlukin-2 (IL-2) and interferon-γ (IFN-γ) (Ko J L, et al. Eur J Biochem. 1995;228:244-9). In another embodiment, the present invention provides a local nasal immunotherapy of a mixture of Dp2 peptide and FIP for allergen-induced airway inflammation and also improves the effectiveness of hyposensitization without adverse effects.

Without intending to limit it in any manner, the present invention will be further illustrated by the following examples.

### EXAMPLE

Materials and methods used herein are described as follows.

Materials and Methods

Animals

Male Balb/c mice were obtained from the National Laboratory Breeding Research Center in Taiwan and were raised in a specific pathogen-free environment. The mice used were between six and eight weeks of age. Groups of six mice were caged separately according to their treatment. Recombinant Dermatophoides pteronyssinus group 2 (rDp2) was prepared as previously described(Tsai J J, et al. Int Arch Allergy Immunol 2000;121:205-10). Dp2 28-40 and Dp2 28-40A were obtained from Dr. Jonathan Lamb (Respiratory Medicine Unit, Edinburgh University, Medical School, Edinburgh, UK) (Verhoef A, and Lamb J R. J Immunol 2000;164:6034-6040). FIP was obtained from Dr. Ko Jiunn-Liang (Institute of Toxicology, Chung Shan Medical University, Taichung, R O C) (J L Ko, et al. Eur. J. Biochem. 1995;228:244-249).

Induction of Allergic Airway Inflammation

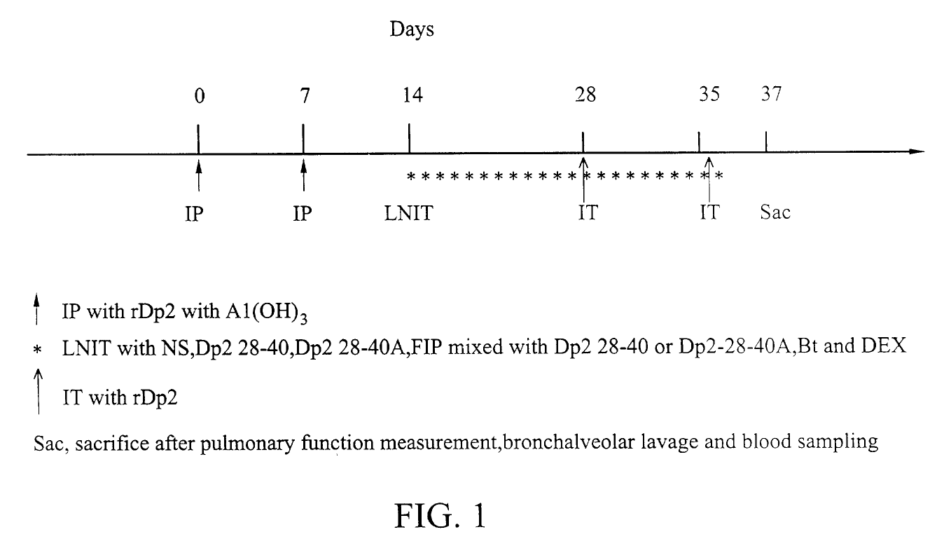

Mice were immunized by way of an intraperitoneal (IP) injection of 1 μg/0.1 mL rDp2 emulsified in 4.0 mg/0.062mL aluminum hydroxide Al(OH)₃ (Whitehall Lab Ltd. Punchbowl, NSW, Australia) on day 0 and day 7. Following immunization, local nasal immunotherapy (LNIT) was conducted daily from day 14 to day 35 with Dp2 28-40, Dp2 28-40A, FIP alone or FIP mixed with either Dp2 28-40 or Dp2 28-40A (FIP, 50 μg/mouse/day; Dp2 peptide, 1 μg/20 μL) in PBS with 10% glycerol. Normal saline (NS) and irrelevant allergen Bt were used as a negative control. The other control group of mice was fed Dexamethasome (DEX) 1 μg/100 μL/mouse. On day 28, mice were lightly anesthetized with an IP injection of 60 mg/kg of sodium pentobarbital (Sigma Chemical Co., St Louis, Mo., U.S.A.) and inoculated intratracheally (IT) with 1 μg/50 μL of rDp 2 on day 28 and day 35. Two days subsequent to the second IT inoculation, the mice were sacrificed following the assessment of their pulmonary function (FIG. 1).

Pulmonary Function Determination

Each mouse was placed inside a barometric plethysmograph (Buxco Electronics, Troy, N.Y.). The plethysmograph has two chambers: one is the main or animal chamber (ID 7.5 cm and 5.5 cm height) and the other is the reference chamber (ID 7.5 cm and 3.5 cm height). A differential pressure transducer was employed to detect the pressure difference between the two chambers. The pressure signal was amplified, digitized via an A/D convert card, and sent to a computer with a BioSystem XA program (Buxco, Electronics, Troy, N.Y.), which sampled and calculated the desired respiratory parameters. These parameters were enhanced pause (Penh), tidal volume, breathing frequency, peak inspiratory flow, peak expiratory flow, end-inspiratory pause, and end-expiratory pause.

Aerosol was generated by placing 5 mL of saline or methacholine (Sigma, 1.56 to 25.00 mg/mL) solution in the cup of an ultrasonic nebulizer (DeVilbiss, Somerset, Pa.). It was delivered via a connecting tube and a three-way connector to the animal chamber of the plethysmograph (Hamelmann E, et al. Am J Respir Crit Care Med. 1997;156:766-75). The median size of the aerosol was approximately 3 μm and the range of the sizes was from 1 to 5 μm, according to the manufacturers information. The aerosol usually filled the chamber within 15-20 sec. At first, each mouse inhaled saline aerosol for 3 min. Then the respiratory parameters were measured for 3 min. Then, inhalation of the saline aerosol was replaced by inhalation of the aerosol of methacholine solution for 3 min. In both cases, the aerosol in the chamber was cleared immediately after the exposure. Respiratory parameters were then measured for 3 min following the inhalation of methacholine. A dose-response curve for methacholine was calculated starting from a low dose of 0.031 mg/dL and moving to a higher dose of 25 mg/dL. There was a 15 min interval between exposures. The percentage changes of Penh obtained from each dose of mechacholine were accumulated area under the curve and the values were calculated as means±SEM. Differences in parameters among groups were analyzed by variance.

BAL Collection and Preparation

Subsequent to pulmonary function measurement having been completed, bronchoalveolar lavage (BAL) was performed using the following procedure. BAL fluids were collected with lmL sterile endotoxin-free saline washes of each lung via the trachea for each mouse. BAL cells were washed once with PBS by centrifugation at 200×g at 4° C. Subsequent to washing, the cells were resuspended in RPMI-1640 (Sigma Chemical Co.). The total leukocyte count in BAL fluids was determined using a hemocytometer. The BAL fluids were aspirated and stored at −70° C. until needed for assay purposes. Subsequent to total leukocyte counting, a cytospin preparation of 100 μL BAL fluids (2×10⁵ cells/mL) was conducted, the cells being cytocentrifuged directly onto glass slides at 350 r.p.m. for five minutes using a Cytospin 3 cytocentrifuge (Shandon Scientific Limited, Astmoor, Runcorn, UK), and then stained with Liu stain (Tonyar Diagnostic Inc, Taipei & Hien, Taiwan). The differential counts were performed for a total of 200 leukocyte cells.

Determination of Der p 2-specific IgG1, IgG2a and IgE Antibodies

Blood was obtained from the retro-orbital venous plexus at the commencement and end of the experimental program. Serum IgE, IgG1 and IgG2a titers of anti-Dp2 antibodies were determined using an enzyme-linked immunosorbent assay (ELISA) technique. Microtiter plates (Nunc Lab, Ill., U.S.A.) were coated with 100 μL Dp2 overnight at a concentration of 0.5 μg/mL and left in a 4° C. refrigerator. Microtiter-plate nonspecific binding sites were blocked with 3% BSA in PBS for two hours at room temperature. Plates were washed with PBS-Tween-20 (PBST) three times and stored at −70° C. prior to subsequent use. Subsequent to the addition of a 1:5 dilution for IgE, a 1:100 dilution for IgG1 and a 1:5 dilution for IgG2a of mice serum, plates were incubated at 4° C. overnight, and then washed three times before the antibody (Horse radish peroxidase-conjugated goat anti-mouse IgE and IgG2a Ab 1:800, IgG1 Ab 1:2,000 Southern Biotech Assoc, Inc, Birmingham, Ala., U.S.A.) was added. Following a one-hour incubation at 37° C. and three washes with PBST, the enzyme substrate (2,2″-Azino-bis(3-ethylbenzothiazolin-6-sulfonic acid diammonium salt; Bio-Rad, USA) was added. The reaction was stopped using 50 μL 4N H₂SO₄ after the reaction had been allowed to continue for 15 minutes, following which the optical density was measured at 450nm using a multiscan spectrophotometer (model A-5682, SLT Lab Instruments, Salzburg, Austria). Results were expressed as ELISA units (EU), one EU being defined as the reciprocal value of the serum dilution that elicited an optical density of 1.0. Such a value was selected so as to always lie within the linear component of the dilution curve. To assure reproducibility, a known serum was included with each test as a standard.

Cytokine Assays

IFN-γ and IL-5 were measured using commercially-available ELISA kits, using a specific mouse monoclonal Ab that recognized different epitopes of the cytokine molecules. The lowest detector range was 10 pg/mL. Given that the BALF was considered to be at too diluted a concentration, IL-4 was not measured.

Cell Culture, Immunofluorescence Staining and Flow-cytometry Analysis

Flow cytometric determination of cytokines present in activated murine T helper cells was conducted according to the technique described by Assenmacher et al in 1994 (Assenmacher M, et al. Flow cytometric determination of cytokines in activated murine T helper lymphocytes: expression of interleukin-10 in interferon-γ and in interleukin-4 expressing cells. Eur J Immunol 1994;24;1097-101). Two-color staining methods were used to analyze IFN-γ and IL-5 expression amongst CD4 cells. Leukocytes derived from peripheral blood (PBL) were stimulated with PMA (50 ng/mL), ionomycin (2 μM) and GolgiStop (Cytofix/Cytoperm Plus Pharmingen San Diego, Calif., U.S.A.) for five hours and then washed twice using PBS. The cells were stained with CD4-PerCP, CD8-FITC or IgG1-FITC (PharMingen) at room temperature (RT) for 30 minutes and then washed. Cells were fixed with cytofix/cytoperm at RT for 30 minutes and then stained with anti-cytokine antibody and IgG2a-PE (PharMingen) at RT for 30 minutes and subsequently washed. Both IgG1-FITC and IgG2a-PE were purchased from the same manufacturer as the anticytokine antibodies and the negative controls. Cells were resuspended in 0.5 mL PBS containing 0.1% w/v sodium azide. Mean fluorescence was measured by means of Becton Diskinson flow cytometry (Becton Diskinson, Calif., U.S.A.). A total of 2,000 cells was analyzed for each sample.

Statistical Analysis

Results were expressed as an arithmetic mean±SEM. Significant difference existed amongst groups. Differences between values prior and subsequent to saline or methacholine exposure were analyzed using a paired t-test. Difference between the saline control and the naive groups was analyzed using an unpaired t-test. Difference was considered significant if p<0.05.

### Example 1

### Local nasal Immunotherapy using Dp2 Peptides

1. Effects of Dp2 28-40 and Dp2 28-40A on Airway Inflammation (Table 1)

Dp2 28-40 showed the best anti-inflammatory effect on airway inflammation. Not only total inflammatory cells, but also macrophages, lymphocytes, neutrophils and eosinophils were decreased when compared to the NS groups (Table 1). Dp2 28-40A had an anti-inflammatory effect but it was weaker than Dp2 28-40. Bt also had a mild anti-inflammatory effect, but only neutrophils and eosinophils were lower.

TABLE 1
Leukocyte subpopulation in the BALF derived from all groups of mice

| | Total cell | Macrophage | Lymphocyte | Neutrophil | Eosinophil |
|---|---|---|---|---|---|
| Naive | 14.3 ± 4.3* | 13.3 ± 4.3* | 1.0 ± 0.0* | 0* | 0* |
| NS | 160.0 ± 23.5 | 56.4 ± 12.7 | 41.4 ± 6.5 | 36.8 ± 2.8 | 25.2 ± 4.5 |
| Dp2 28-40 | 53.3 ± 4.1* | 31.0 ± 2.5* | 11.8 ± 0.8* | 7.2 ± 1.5* | 3.5 ± 0.5* |
| Dp2 28-40 A | 65.0 ± 3.2* | 38.4 ± 7.2* | 14.6 ± 3.3* | 8.2 ± 1.1* | 3.0 ± 1.0* |
| Bt | 111.2 ± 13.4* | 49.0 ± 7.5 | 28.2 ± 1.9 | 21.0 ± 3.0* | 14.8 ± 3.3* |
| DEX | 46.4 ± 2.2* | 29.2 ± 4.4* | 9.4 ± 1.5* | 6.6 ± 1.0* | 3.2 ± 0.4* |

*p < 0.05 compared with normal saline treatment.
10⁴ cells/mL, data represented as mean ± SEM of six animals.

2. Effects of Dp2 28-40 and Dp2 28-40A on Cytokine Producing Cells in the Peripheral Blood Mononuclear Cells (Table 2)

In the NS treated mice, the percentage of IL-5⁺/CD4⁺ cells were higher and IFN-γ⁺/CD4⁺ and IFN-γ⁺/CD8⁺ cells were lower than naive mice (Table2). These alterations of T cell subpopulations were abolished by the treatment of Dp2 28-40 and Dp2 28-40A. Although Bt LNIT can up-regulate IFN-γ⁺/CD4⁺ cells, the increase was trivial with the ratio of IL-5/IFN-γ remaining similar to the NS control. When the two peptides were compared, Dp2 28-40 had a more potent effect than Dp2 28-40A on the increase in IFN-γ and IL-5 producing CD4 cells and CD8 cells. However, the ratio of IL-5/IFN-γ remained similar between these two groups (Table2).

TABLE 2
Effect of Dp2 28-40 and Dp2 28-40A on cytokine
producing cells in peripheral blood^{a}

| | Naive | NS | Dp2 28-40 | Dp2 28-40A | Bt | DEX |
|---|---|---|---|---|---|---|
| CD4⁺ | 43.4 | 50.3 | 56.0 | 51.8 | 44.2 | 45.6 |
| IL-5⁺/CD4⁺ | 15.4 | 26.3 | 17.7 | 11.4 | 34.2 | 9.2 |
| IFN-γ⁺/CD4⁺ | 17.9 | 9.2 | 27.8 | 11.4 | 27.0 | 7.9 |
| IL-5/IFN-γ | 0.9 | 2.9 | 0.6 | 1.0 | 1.3 | 1.2 |
| (CD4) | | | | | | |
| CD8⁺ | 7.6 | 9.6 | 9.8 | 10.7 | 9.1 | 7.0 |
| IL-5⁺/CD8⁺ | 9.5 | 5.5 | 9.5 | 3.5 | 17.1 | 8.2 |
| IFN-γ⁺/CD8⁺ | 15.7 | 4.9 | 16.8 | 5.8 | 13.0 | 7.9 |
| IL-5/IFN-γ | 0.6 | 1.1 | 0.6 | 0.6 | 1.3 | 1.0 |
| (CD8) | | | | | | |

^{a}Data represented as percentage of positive stained cells in the PBMC of six mice.

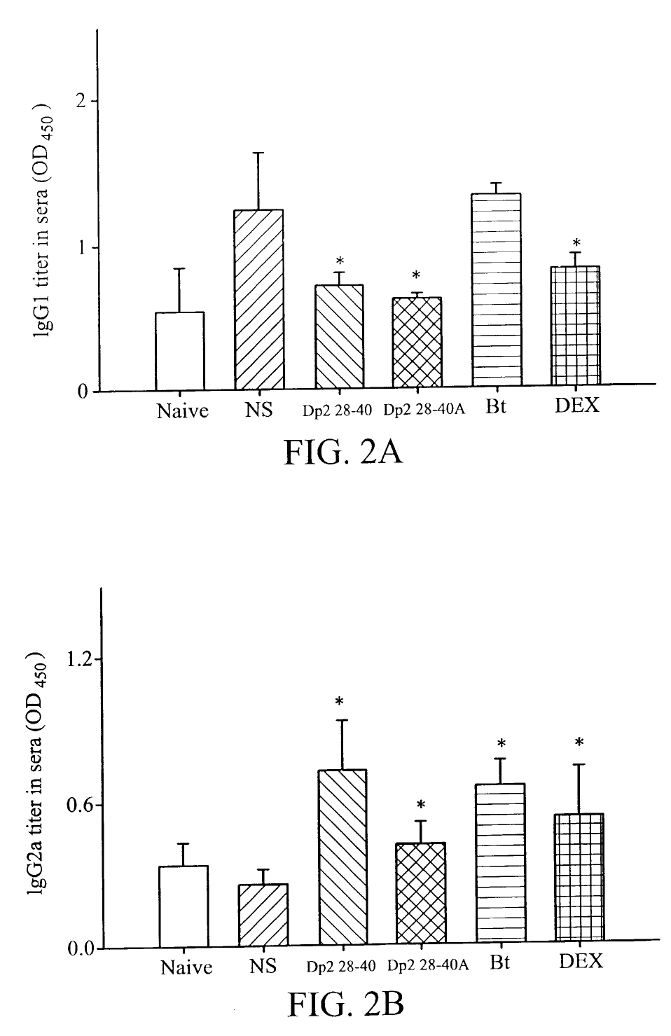

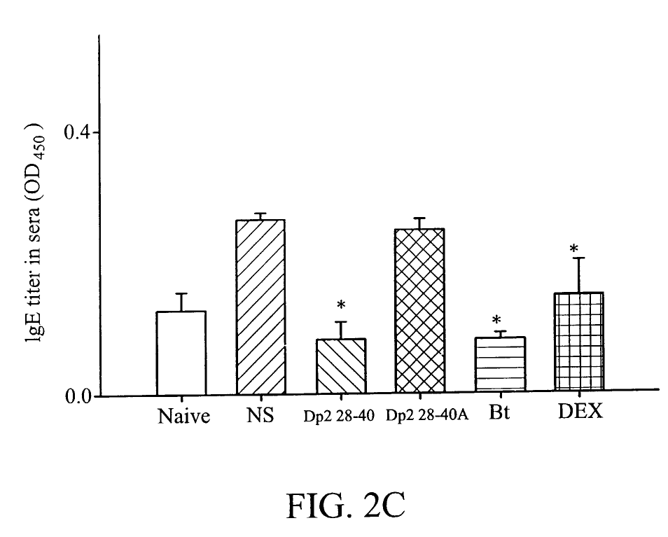

3. Effects of Dp2 28-40, and Dp2 28-40A on Dp2 Specific IgG1 (FIG. 2A), IgG2a (FIG. 2B) and IgE (FIG. 2C) Production in the Sera

In the NS treated mice, both IgG1 and IgE were significantly higher in the sera when compared with naive control; however, there were no significant changes in IgG2a level. In the groups of LNIT with Dp2 28-40 and Dp2 28-40A, the IgG1 was significantly reduced and the IgG2a was increased in comparison with that of the NS group. However, the reduction of IgE was observed only in the group of Dp2 28-40. In the Bt treated mice, there were also significant increases in IgG2a and reduction of IgE and no significant change in IgG1. Although the changes of antibody production were treated in the group of Dp2 28-40, similar findings were observed in the group of DEX.

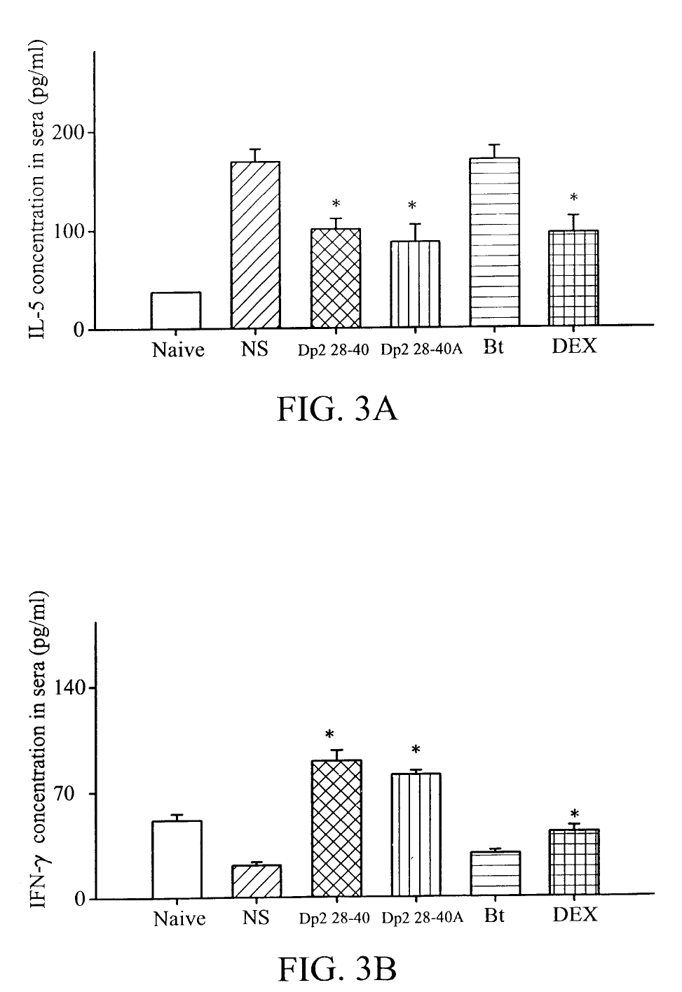

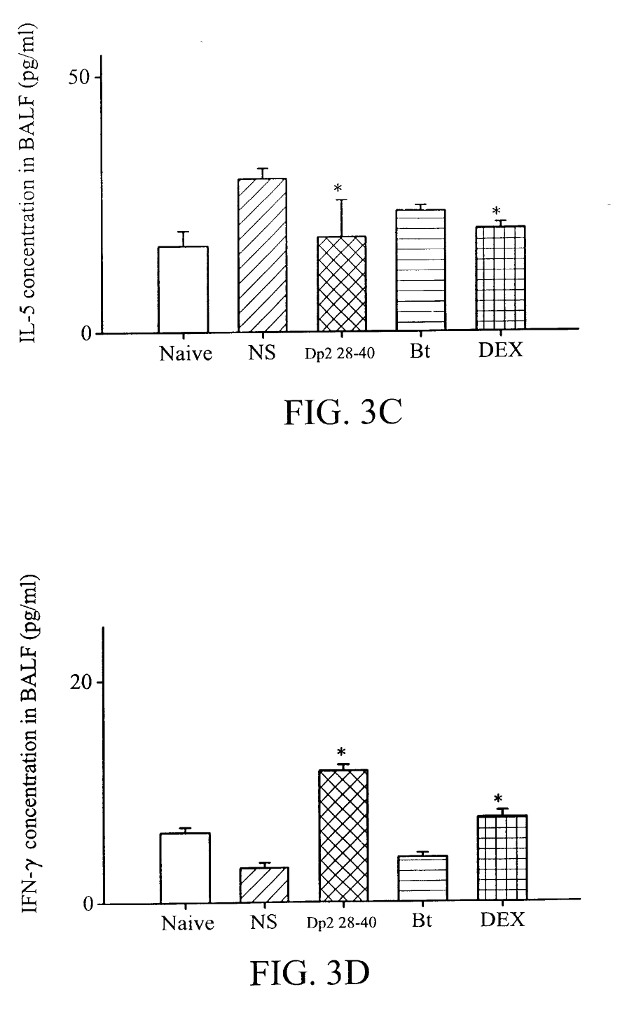

4. Effects of Dp2 28-40 and Dp2 28-40A on Cytokine Levels in Sera and BALF (FIG. 3A-3D)

Significantly increased IFN-γ and decreased IL-5 were observed in the sera for both the Dp2 28-40 and Dp2 28-40A therapy groups compared to the NS treated mice. When the effects of Dp2 28-40 and Dp2 28-40A were compared, the reduction in IL-5 and the increase in IFN-γ were similar in the two groups (FIG. 3A-3B). In the Bt treated mice, there was no significant difference in either IL-5 or IFN-γ compared to the NS treated group. In BAL fluid, there was a significant reduction in IL-5 and a significant increase in IFN-γ after Dp2 28-40 LNIT (FIG. 3C-3D). There were no statistically significant differences in cytokine levels between Bt- and NS-treated mice.

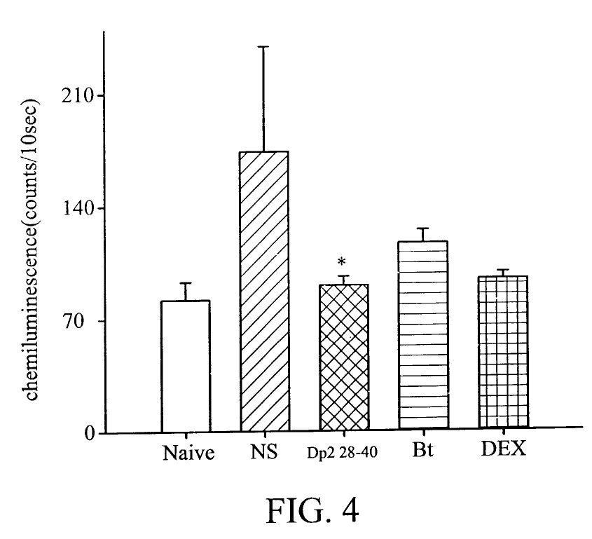

5. Effects of Dp2 28-40 on Free Radical Production (FIG. 4)

Using chemiluminescence, free radicals in the BAL fluid were measured immediately after each experiment. The total amounts of free radical were detected and expressed as counts/10 seconds. Results showed that Dp2 28-40 therapy significantly inhibited free radical formation in the airway compared to the NS control. Although Bt therapy also had an inhibitory effect, it did not reach a statistically significant level (FIG. 4).

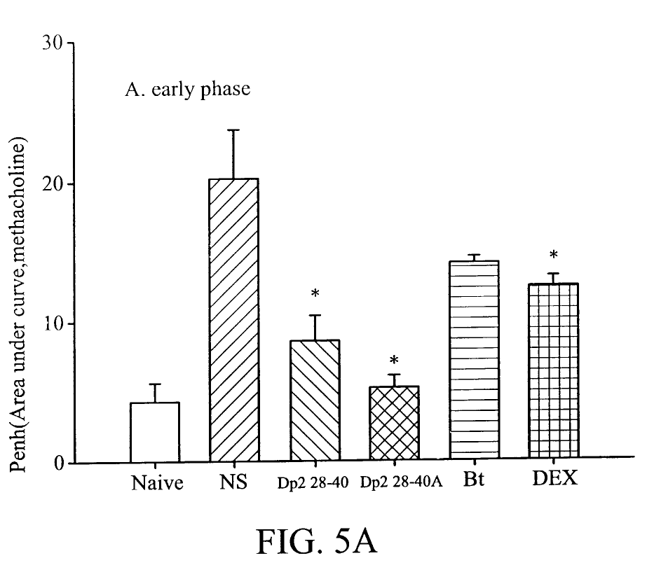

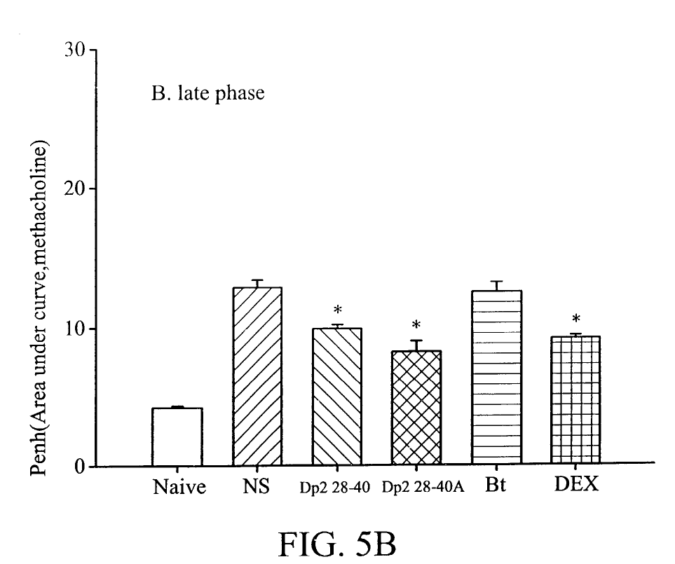

6. Effects of Dp2 28-40 and Dp2 28-40A on the Pulmonary Function (FIGS. 5A-5B)

The airway hypersensitivity to methacholine was measured both 30 min (early phase, FIG. 5A) and 24hrs (late phase, FIG. 5B) after rDp2 challenge. In the Dp2 28-40 and Dp2 28-40A LNIT mice, the Penh significantly decreased after methacholine challenge in both early and late phase after rDp2 intratracheal challenge compared to the NS treated mice. A similar change in Penh was observed between Dp2 28-40 and Dp2 28-40A group. In the Bt treated group, the decrease of Penh was trivial and did not reach a statistically significant level.

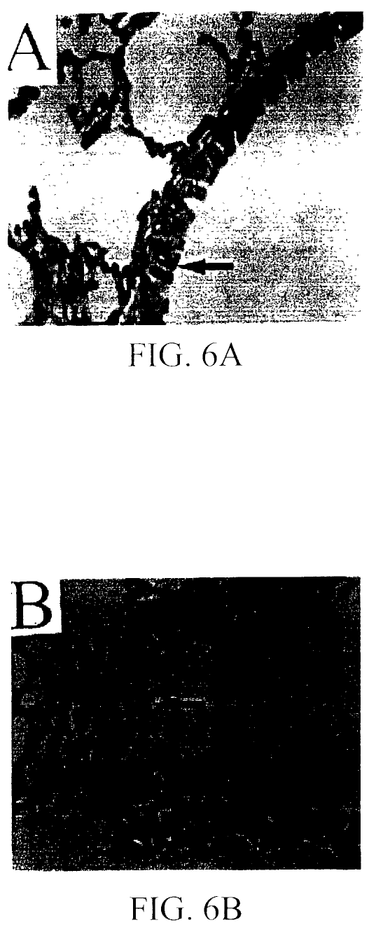

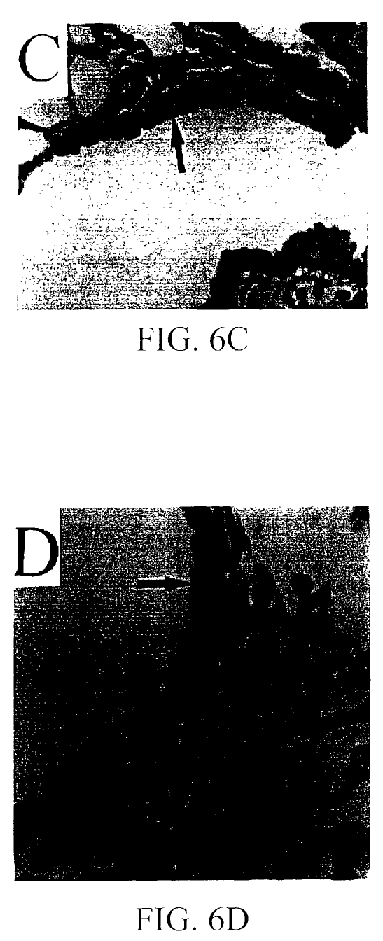

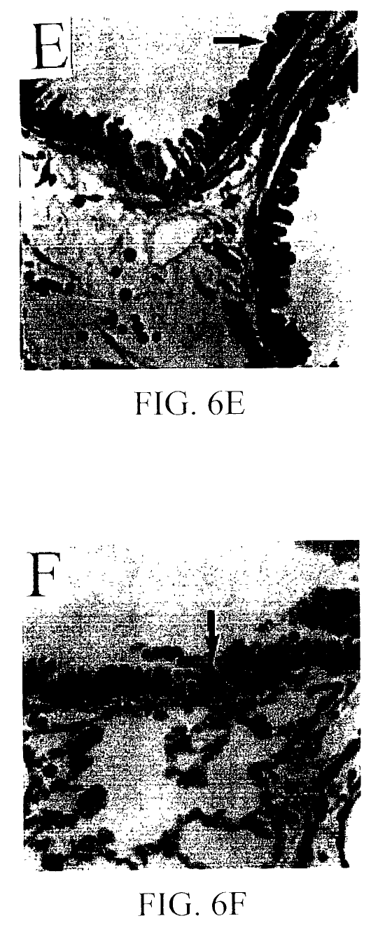

7. The Pathology of Different Groups of Mice after LNIT (FIGS. 6A-6F)

Effects of rDp2 sensitization and challenge in the inflammatory cell infiltration and epithelium damage in the airway were compared after LNIT. The results showed that there were reductions in airway inflammation in both Dp2 28-40 (FIG. 6D) and Dp2 28-40A (FIG. 6E).

8.Discussion

In Example 1, it was demonstrated that rDp2-induced airway inflammation could be abolished by LNIT with Dp2 28-40 and Dp2 28-40A. BALF analysis showed that not only the total cell numbers but also the level of various inflammatory cells was decreased after therapy. This indicated that LNIT with either Dp2 28-40 or Dp2 28-40A has a potent anti-inflammatory effect.

The inhibitory effect on Dp2 specific IgGl and IgE by Dp2 28-40, but not Dp2 28-40A, suggests that Dp2 28-40 nasal administration has a more potent modulatory effect on immunoglobulin synthesis.

It was also found that LNIT was associated with an increase in IFN-γ and a decrease in IL-5 in both BALF and sera, indicating that both peptide nasal immunotherapies upregulated the Th1 and down-regulated the Th2 cytokines. However, Bt did not show any significant effect on cytokine concentration either in the BALF or sera, suggesting that the peptide epitope interaction is more specific and important for cytokine production. In addition, it was demonstrated that Dp2 28-40, as well as Dp2 28-40A, increase both the IFN-γ⁺/CD4⁺ and IFN-γ⁺/CD8⁺ cells to a similar extent. The peptides also had a similar inhibitory effect on IL-5 producing CD4 cells. The results suggest that the peptides are specific to T cell activation. Despite the similar extent of T cell activation, there is a discrepancy between Dp2 28-40 and Dp2 28-40A at the level of immunoglobulin synthesis.

Abnormal non-allergic airway hyperresponsiveness is viewed as a characteristic of airway inflammatory reaction (Fish J E, Shaver J R, Peters S P. Airway hyperresponsiveness in asthma. Is it unique? Chest. 1995; 107:154S-156S). rDp2induced airway hyperreactivity to methacholine and inflammatory cell infiltration into the airway can be abolished by either Dp2 28-40 or Dp2 28-40A. This indicates that the protective effect of LNIT on airway hypersensitivity may be due to a reduction in airway inflammation.

Moreover, rDp2-induced airway inflammation can be down-regulated by both Dp2 28-40 and Dp2 28-40A. This phenomenon suggests that peptide administration may modulate the immune response caused by the whole allergen.

In conclusion, Example 1 of the present invention has demonstrated that LNIT with a single allergen derived peptide or its analog attenuate whole-allergen-induced airway inflammation and airway hyperresponsiveness. This anti-inflammatory effect may be through up-regulation and inhibition of allergen specific IgE production by B cells. The data suggests that up-regulation of Th1-type cytokine (IFN-γ) by allergen-derived peptide nasal administration may provide a useful alternative to specific immunotherapy for the treatment of atopic allergic disease.

### Example 2

### Local Nasal Immunotherapy using a Mixture of Dp2 Peptide Epitopes and Fungal Immunomodulatory Protein

1. Effects of FIP and a Mixture of FIP with Dp2 28-40 or Dp2 28-40A upon the Inflammatory Cells Obtained from BALF

The anti-inflammatory effects of FIP and a mixture of FIP with Dp2 28-40 or Dp2 28-40A upon rDp2-immunized mice were determined by analyzing the cellular component in the derived BALF. The results revealed that FIP was able to diminish the airway-inflammation level; not only did the total leukocyte count decrease, but also the same was the case for the level of eosinophils, neutrophils and lymphocytes subsequent to LNIT. Similar findings were detected for the groups comprising a mixture of FIP with Dp2 28-40 and a mixture of FIP with Dp2 28-40A. A decrease in macrophage level was only observed for the groups for which the FIP was combined with Dp2 28-40 or 28-40A (Table 3).

TABLE 3
Effects of FIP and FIP mixed with Dp2 epitope
peptides on the inflammatory cells obtained from BALF

| | Total cell | Macrophage | Lymphocyte | Neutrophil | Eosinophil |
|---|---|---|---|---|---|
| Naive | 13.3 ± 0.9* | 12.3 ± 0.8* | 1.0 ± 0.0* | 0* | 0* |
| NS | 115.0 ± 2.1 | 35.4 ± 2.3 | 27.0 ± 1.0 | 30.3 ± 1.4 | 22.0 ± 2.0 |
| FIP | 65.0 ± 10.5* | 37.0 ± 7.4 | 13.0 ± 1.8* | 9.0 ± 2.4* | 6.3 ± 1.3* |
| FIP + Dp2 28-40 | 61.0 ± 1.5* | 27.7 ± 2.1* | 20.6 ± 0.6* | 9.3 ± 2.0* | 4.0 ± 0.6* |
| FIP + Dp2 28-40A | 61.0 ± 1.5* | 23.3 ± 1.8* | 16.7 ± 0.9* | 13.7 ± 0.9* | 7.3 ± 0.3* |
| DEX | 55.5 ± 1.7 | 28.3 ± 1.7* | 15.3 ± 0.6* | 8.0 ± 0.8* | 3.8 ± 0.5* |

Data represented as mean ± SEN of six mice. (×10⁴/mL)
*P < 0.05 compared with NS control.

2. Effects of FIP and a Mixture of FIP with Dp2 28-40 or Dp2 28-40A upon Cytokine-Producing Cells in Peripheral Blood

The effects of FIP alone, and a combination of FIP with Dp2 28-40 or Dp2 28-40A upon cytokine-producing cells were compared following LNIT. Both CD4 and CD8 were assessed for cytokine production. The results revealed that FIP can down-regulate cytokine-producing CD4 and CD8 cells. Both IFN-Υ and IL-5-producing cell levels decreased subsequent to LNIT with FIP when compared with the corresponding level for the NS control. When the IL-5/IFN-γ ratio was analyzed, it was noted that the ratio of IL-5/IFN-γ also decreased, but only in the presence of CD4 cells. When Dp2-immunized mice were treated with a mixture of FIP with either Dp2 28-40 or Dp2 28-40A, the percentage of IL-5 producing CD4 and CD8 cells was noted to decreased, but IFN-γ-producing cell levels increased. The IL-5/IFN-γ ratio was also noted to decrease for both CD4 and CD8 cells (Table 4).

TABLE 4
Effects of FIP and FIP mixed with Dp2 epitope
pepteides on cytokine producing cells in peripheral blood

| | | | | FIP + | FIP + | |
|---|---|---|---|---|---|---|
| | Naive | NS | FIP | Dp2 28-40 | Dp2 28-40A | DEX |
| CD4 | 50.8 | 55.5 | 29.1 | 49.3 | 59.7 | 51.2 |
| CD8 | 6.4 | 4.9 | 7.0 | 8.9 | 3.0 | 7.9 |
| IL-5⁺/CD4⁺ | 17.0 | 35.9 | 12.6 | 19.4 | 15.7 | 15.8 |
| IFN-γ⁺/CD4⁺ | 22.1 | 16.5 | 13.7 | 28.2 | 22.7 | 17.7 |
| IL-5/IFN-γ | 0.8 | 2.2 | 0.9 | 0.7 | 0.7 | 0.9 |
| (CD4) | | | | | | |
| IL-5⁺/CD8⁺ | 10.4 | 11.6 | 8.4 | 10.5 | 5.8 | 14.1 |
| IFN-γ⁺/CD8⁺ | 16.2 | 7.2 | 5.0 | 14.03 | 12.9 | 14.1 |
| IL-5/IFN-γ | 0.6 | 1.6 | 1.7 | 0.8 | 0.4 | 1.0 |
| (CD8) | | | | | | |

*Data represented as percentage of total leukocytes in the peripheral blood of six mice.

3. Effects of FIP and a Mixture of FIP with Dp2 28-40 or Dp2 28-40A upon Cytokine Profile in the Sera and BALF.

To determine the effect of FIP and a mixture of FIP with Dp2 28-40 or Dp2 28-40A upon cytokine production subsequent to LNIT, both sera and BALF were obtained for IFN-γ and IL-5 determination. The results showed that FIP was able to elicit a decrease in IL-5 level and also to increase IFN-γ production in both sera and BALF. When Dp2 28-40 or Dp2 28-40A were combined with FIP, the modulatory effect was altered. The concentration of IL-5 and IFN-γ were higher than that of FIP alone in both sera and BALF, and it was noted that IL-5 levels were lower and IFN-Υ levels higher for the group treated with a combination of FIP and Dp2 28-40 by comparison with that of its corollary combination, FIP mixed with Dp2 28-40A (Table 5).

TABLE 5
Effects of FIP and FIP mixed with Dp2 epitope
peptides on cytokine profile in the sera and BALF

| | | | | FIP + | FIP + | |
|---|---|---|---|---|---|---|
| | Naive | NS | FIP | Dp2 28-40 | Dp2 28-40A | DEX |
| Sera | | | | | | |
| IL-5 | 29.4 ± 1.0* | 189.3 ± 13.4 | 77.6 ± 1.9* | 98.2 ± 1.9* | 125.0 ± 6.6* | 138.0 ± 16.6* |
| IFN-γ | 48.5 ± 3.9* | 21.1 ± 2.6 | 52.0 ± 1.9* | 76.0 ± 1.9* | 69.8 ± 2.0* | 46.2 ± 3.4* |
| BALF | | | | | | |
| IL-5 | 15.2 ± 2.9* | 34.8 ± 1.9 | 19.3 ± 1.9* | 20.3 ± 1.9* | 25.9 ± 3.2* | 19.2 ± 1.2* |
| IFN-γ | 8.3 ± 0.7* | 4.2 ± 0.4 | 8.3 ± 0.4* | 12.3 ± 0.4* | 11.8 ± 2.2* | 9.9 ± 0.6* |

Data represent as mean ± SEM (pg/mL). (n = 6)
*P < 0.05 compared with NS control.

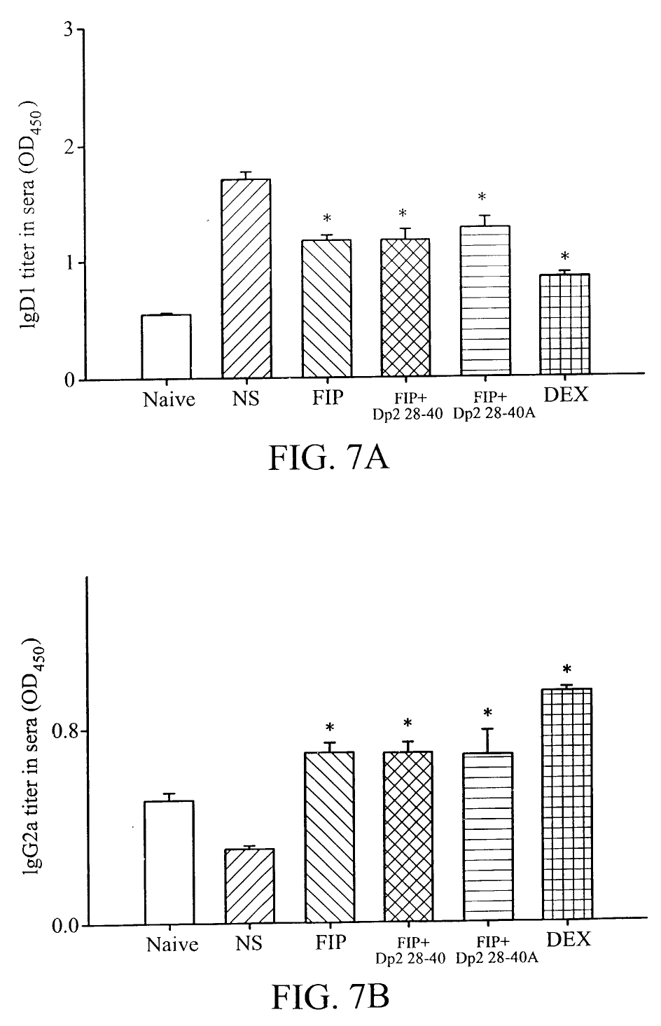

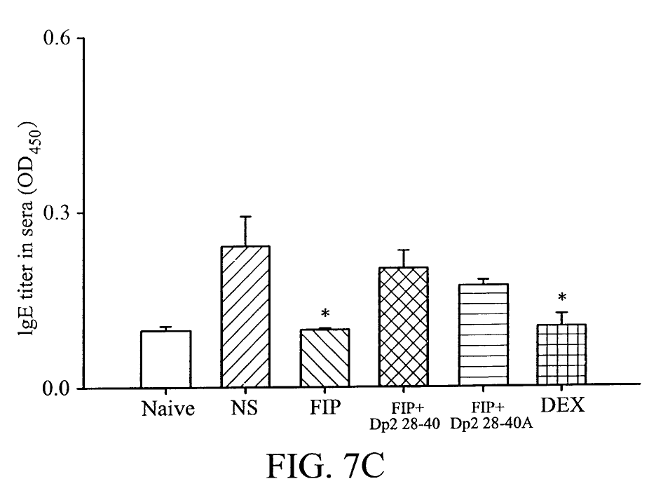

4. Effects of FIP and a Mixture of FIP with Dp2 28-40 or Dp2 28-40A upon Dp2 Specific IgG1 (FIG. 7A), IgG2a (FIG. 7B) and IgE (FIG. 7C) Production in Sera.

For the rDp2-immunized mice, following their exposure to LNIT with NS, both IgG1 and IgE levels remained elevated by comparison to that of naive mice. Both IgG1 and IgE levels were significantly reduced and IgG2a levels were significantly increased when mice were treated LNIT with FIP. The IgG1 levels were significantly reduced and IgG2a levels were significantly increased for both FIP mixture with Dp2 28-40 and FIP mixed with Dp2 28-40A. The modulatory effect of FIP mixed with Dp2 28-40 or Dp2 28-40A upon IgE levels were trivial although the effect of FIP upon IgE level was Dp2 28-40 or Dp2 28-40A significantly reduced.

5. Effects of FIP and FIP Mixed with Dp2 28-40 and Dp2 28-40A upon Pulmonary Function.

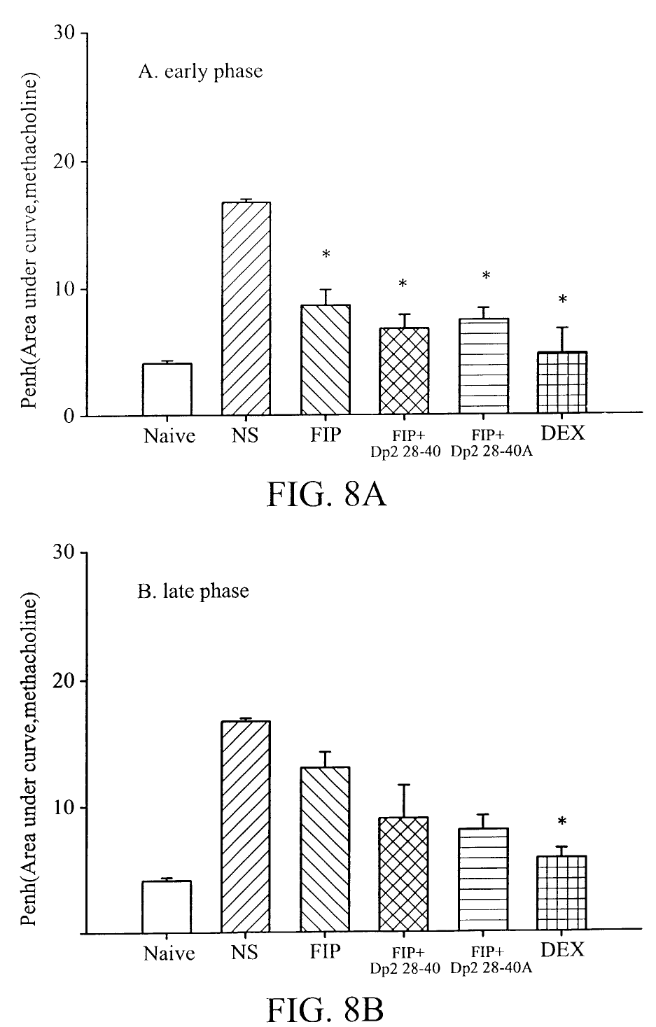

In the FIP and FIP mixed with Dp2 28-40 and Dp2 28-40A LNIT mice, the penh significantly decreased compared to NS treated mice 30 min after rDp2 intratracheal challenge (FIG. 8A). Although similar decrease of penh was observed in the late phase, the decrease of penh did not reach a statistically significant level (FIG. 8B).

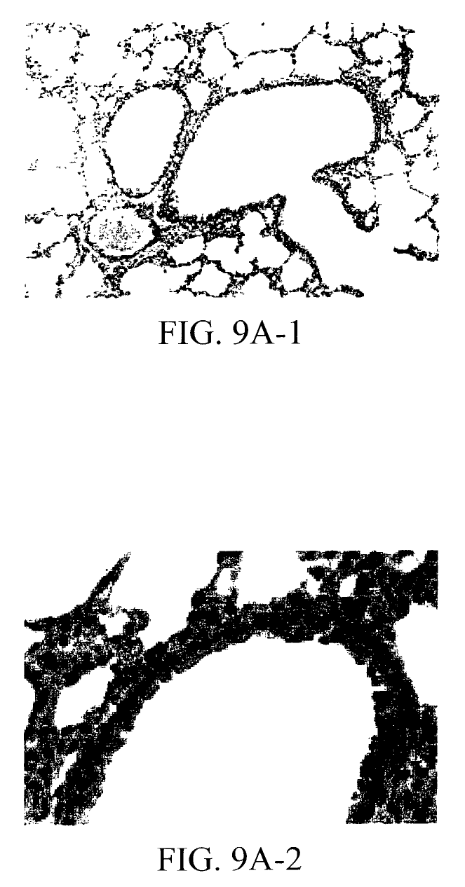

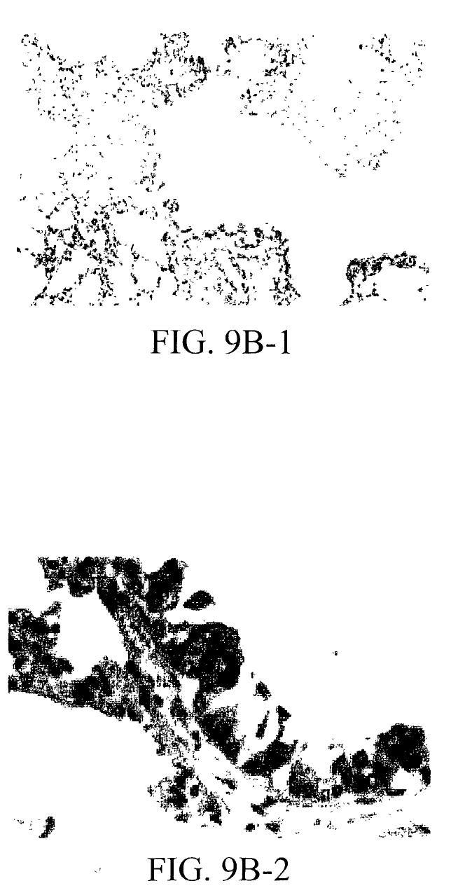

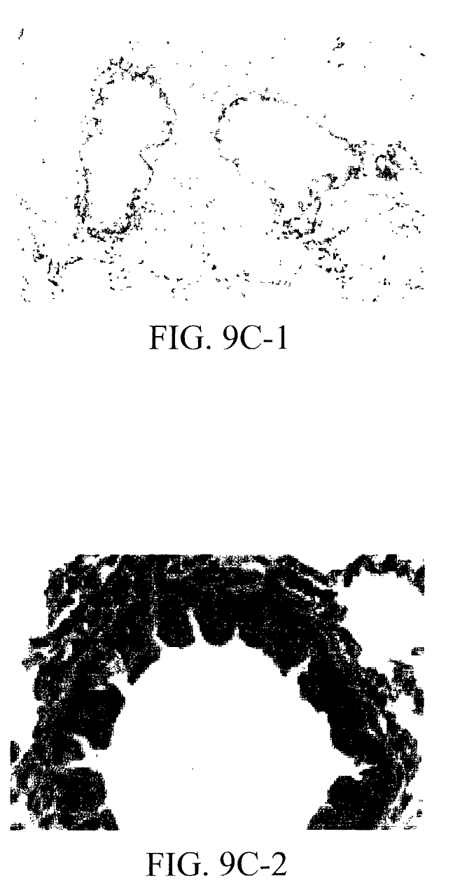

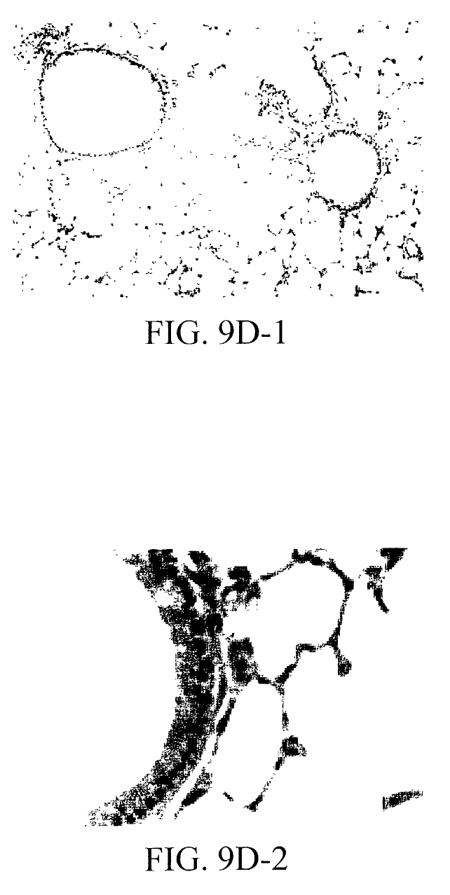

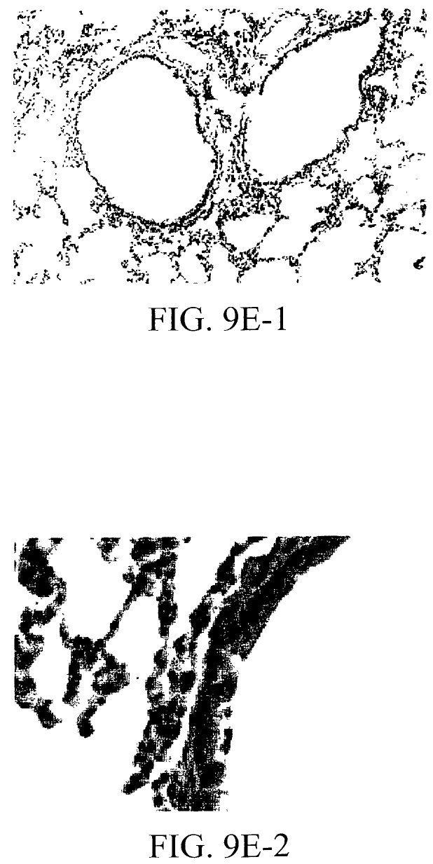

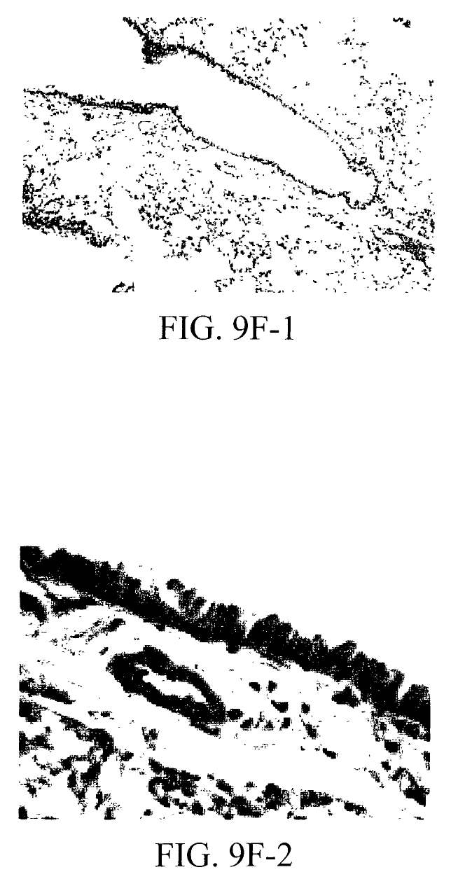

6. The Pathology of Different Groups of Mice after LNIT (FIGS. 9A-9F)

The effect of rDp2 sensitization and challenge in the context of inflammatory-cell infiltration and epithelial damage in the airways of test mice were compared subsequent to LNIT, the results revealing that there was a reduction in the level of airway inflammation for both FIP (FIG. 9D) and FIP mixed with Dp2 28-40 (FIG. 9F) or FIP mixed with Dp2 28-40A (FIG. 9E).

7. Discussion

In Example 2 of the present invention, it was demonstrated that a dominant peptide of the Th2 response by rDp2-immunized mice can be modulated by LNIT with the Dp2 peptide epitope mixed with FIP. LNIT using FIP mixed with the dominant peptide of Dp2 28-40 or the altered peptide Dp2 28-40A was noted to be able to suppress on-going airway inflammation whilst rDp2-induced airway hyperreactivity. This LNIT-induced suppression of airway hyperreactivity was associated with an increase in IFN-γ production and an increase in the level of the Th1-derived IgG2a antibody subclass. These data suggest that the mixture of FIP with an allergen-derived peptide can be used to modulate the allergen-induced airway inflammation through Th1 activation. As has been noted above an enhanced IgG2a and reduced IgG1 responses occur following LNIT with either FIP alone or a combination of FIP with Dp2 allergic epitope peptides, the modulation of IgE response does not occur following LNIT with a combination of FIP with Dp2 allergic epitope peptides. These results suggest that the in-vivo immunomodulatory properties of the B-cell IgE reaction to FIP with Dp2 allergic epitope peptides were somewhat trivial and less effective than B-cell IgG reaction. Due to the mixture of FIP with Dp2 28-40 and FIP with Dp2 28-40A for a more-effective and safer immunotherapy procedure, comparison of their effects via LNIT upon mice primed with rDp2 in aluminum hydroxide induces a pre-existing Th2 response. The relatively high level of IFN-γ and the corresponding relatively low level of IL-5 in both BALF and sera suggest that LNIT could transform allergen-specific T cells, changing from a Th2 to a Th1-like type, this process possibly being similar to subcutaneous immunotherapy.

Although LNIT with FIP also indicated a shift in the Th1 and Th2 response for test mice based upon the results of cytokine, it was weaker than that induced as a result of subjecting test mice to a mixture of FIP and Dp2 epitope peptides which resulted in a decrease in the IL-5/IFN-γ ratio for both CD4 and CD8 cells. The value of the ratio of IL-5/IFN-γ for CD8 cells was observed to be greater for the test group FIP mixed with Dp2 28-40 than that for FIP mixed with Dp2 28-40A. The results reveal that there were fewer IL-5-secretory CD4 and CD8 cells for those mice treated with a mixture of FIP with Dp2 28-40A than was the case for test mice exposed to a combination of FIP and Dp2 28-40. The results also suggest that Dp2 28-40A is more effective at downregulating Th2 cells.

Similarly to subcutaneous immunotherapy, LNIT of allergen carries the risk of inducing anaphylatic reactions, such adverse reactions being one of the principal reasons that immunotherapy is used less today than in the past. T-cell-targeted strategies as a result of the development of non-anaphylactic allergens have been previously used for subcutaneous immunotherapy regimens, although due to the sometimes sinister nature of late-occurring side effects, such procedures have largely been discontinued, although this form of treatment utilizing a short peptide has been recently reassessed and phase II trials are ongoing. And such appear to be not reactive with IgE and not to release histamine from mast cells. This may suggest that LNIT with short peptides might be safer at even a higher dose than is currently used.

In summary, these studies have demonstrated that FIP in combination with Dp2 28-40 or 28-40A makes a good alternative candidate for human immunotherapy. This combination of enhanced immunogenicity and reduced allergenicity to an antigen preparation and the relatively easy and safe nasal application method for this therapeutic method are encouraging for the development of an appropriate mixture of FIP with altered allergenic epitope peptide to constitute an efficacious mode of allergen immunotherapy.

While the invention has been particularly shown and described with the reference to the preferred embodiment thereof, it will be understood by those skilled in the art that various changes in form and details may be made without departing from the spirit and scope of the invention.

What is claimed is:

1. A method for treating allergen-induced airway inflammation, comprising the steps of:
administering a therapeutically effective amount of a pharmaceutical composition comprising a Dermatophoides pteronyssinus group 2 (Dp2) epitope peptide and a pharmaceutically acceptable carrier to an individual having allergen-induced airway inflammation in a manner consistent with local nasal immunotherapy.

2. The method as claimed in claim 1, wherein the allergen-induced airway inflammation comprises dust mite allergic asthma, allergic rhinitis, or nasosinusitis.

3. The method as claimed in claim 1, wherein the pharmaceutical composition is in the form of nasal drops, nasal spray, cream or nasal strip.

4. The method as claimed in claim 3, wherein the nasal strip is nitrocellulose (NC), polyvinylidene fluoride (PVDF), nylon, filter papers, fabric, cloth, polyethylene, polypropylene, composite fibers, flexible medical grade materials, or the combination thereof.

5. The method as claimed in claim 1, wherein the Dermatophoides pteronyssinus group 2 epitope peptide id Dp2 28-40 or Dp2 28-40A, wherein Dp2 28-40 is encoded from amino acid sequence ID No. 1 and Dp2 28-40 A is encoded from amino acide sequence ID No. 2.

6. The method as claimed in claim 1, wherein the compositioon further comprises a fungal immunomodulatory protein (FIP) isolated from Flammalina velutipes, wherein the FIP is encoded from amino acid sequence ID No. 3.

7. The method as claimed in claim 5, wherein the Dermatophoides pteronyssinus group 2 epitope peptide is in a dosage of 0.1˜10 μg.

8. The method as claimed in claim 7, wherein the Dermatophoides pteronyssinus group 2 epitope is in a dosage of 1 μg.

9. The method as claimed in claim 6, wherein the FIP is in a dosage of 1˜100 μg.

10. The method as claimed in claim 9, wherin the FIP is in a dosage of 50 μg.

11. A pharmaceutical composition for treating allergen-induced airway inflamation in an individual, comprising:
a Dermatophoided pyeronssinus group 2 )Dp2) epitope peptide; and
a pharmaceutically acceptable carrier.

12. the pharmaceutical composition as claimed in claim 11, wherein the allergen-induced airway inflamation comprises dust mite allergic asthma, allergic rhinitis, or nasosinusitis.

13. The pharmaceutical composition as claimed in claim 11, wherein the pharmaceutical composition is in the form of nasal drops, nasal spray, cream or nasal strip.

14. The pharmaceutical composition as claimed in claim 13, wherein the nasal strip is nitrocellulose (NC), polyvinylidene fluoride (PVDF), nylon, filter papers, fabric, cloth, polyethylene,polypropylene, composite fibers, flexible medical grade materials, or the combination thereof.

15. The pharmaceutical composition as claimed in claim 11, wherein the Dermatophoides pteronyssinus group 2 epitope peptide is Dp2 28-40 or Dp2 28-40A, wherein Dp2 28-40 is encoded from amino acid sequence ID No. 1 and Dp2 28-40A is encoded from amino acid sequence ID No. 2.

16. The pharmaceutical composition as claimed in claim 11, wherein the composition further comprises a fungal immunomodulatory protein(FIP) isolated from Flammalina velutipes, wherein the FIP is encoded from amino acid sequence ID No. 3.

17. The pharmaceutical composition as claimed in claim 15, wherein the Dermatophoides pteronyssinus group 2 epitope peptide is in a concentration of 0.1˜10 μg/20 μl.

18. The pharmaceutical composition as claimed in claim 17, wherein the Dermatophoides pteronyssinus group 2 epitope peptide is in a concentration of 1 μg/20 μl.

19. The pharmaceutical composition as claimed in claim 16, wherein the FIP is in a concentration of 1˜100 μg/20 μl.

20. The pharmaceutical composition as claimed in claim 19, wherein the FIP is in a concentration of 50 μg/20 μl.
